(19) United States
(12) Plant Patent Application Publication
(10) Pub. No.: US 2026/0026416 P1
(43) Pub. Date: Jan. 22, 2026

(54) Miniature Rose Plant Named 'Poulty054'

(50) ;

(71) Applicant: Mogens Nyegaard Olesen, Fredensborg (DK)

(72) Inventor: Mogens Nyegaard Olesen, Fredensborg (DK)

(73) Assignee: Poulsen Roser A/S, Fredensborg (DK)

(21) Appl. No.: 18/831,809

(22) Filed: Sep. 15, 2025

(30) Foreign Application Priority Data
Sep. 19, 2021 (QZ) 20242196

(51) Int. Cl. A01H 6/74; A01H 5/02

(52) U.S. Cl. CPC A01H 6/749; A01H 5/02
USPC PLT/119

(57) ABSTRACT

A new garden rose plant of the miniature class which has abundant, orange-yellow bicolor flowers and attractive foliage. This new and distinct variety has shown to be uniform and stable in the resulting generations from asexual propagation.

Botanical designation: Rosa hybrida.

Variety denomination: ‘Poulty054’.

This application claims priority to Plant Breeder's Rights Application Number 2024/2196, which was filed at the Community Plant Variety Rights Office in the European Union on Sep. 19, 2024, the contents of which are hereby incorporated by reference for all purposes.

### SUMMARY OF THE INVENTION

The present invention constitutes a new and distinct variety of rose plant which originated from a controlled crossing between the female seed parent, an unnamed seedling, and the male pollen parent, also an unnamed seedling. Both of the parent varieties are non-patented.

The two parents were crossed during the summer of 2020 and the resulting seeds were planted in a controlled environment in Odense, Denmark. The new variety, named ‘Poulty054’, originated as a single seedling from the stated cross.

The new variety may be distinguished from its male pollen parent and female seed parent primarily by the following characteristics. The male pollen parent plant has yellow flowers while the new variety has orange-yellow bicolor flowers. The female seed parent plant has red flowers while the new variety has orange-yellow bicolor flowers.

The objective of the hybridization of this rose variety was to create a new and distinct variety with unique qualities, such as:
1. Uniform and abundant orange-yellow bicolor flowers with long lasting shelf life and stabilty;
2. Vigorous, but very compact growth when propagated on its own roots;
3. Suitability for commercial production in small containers; and
4. Exceptional disease resistance.

This combination of qualities is not present in previously available commercial cultivars of this type, known to the inventor, and distinguish ‘Poulty054’ from all other varieties of which we are aware.

As part of the rose development program, Mogens N. Olesen germinated the seeds from the aforementioned hybridization during winter of 2020 and conducted evaluations on the resulting seedlings in a controlled environment in Odense, Denmark. ‘Poulty054’ was selected in the spring of 2021 by the inventor as a single plant from the progeny of the aforementioned hybridization.

Asexual reproduction of ‘Poulty054’ by rooted cuttings was first done by Mogens N. Olesen in the nursery in Odense, Denmark in July 2021. This initial and other subsequent asexual propagations conducted in controlled environments have demonstrated that the characteristics of ‘Poulty054’ are true to type and are transmitted from one generation to the next.

### DESCRIPTION OF THE DRAWING

The accompanying color illustrations show as true as is reasonably possible to obtain in color photographs of this type, the typical characteristics of the buds, flowers, leaves, and stems, of ‘Poulty054’.

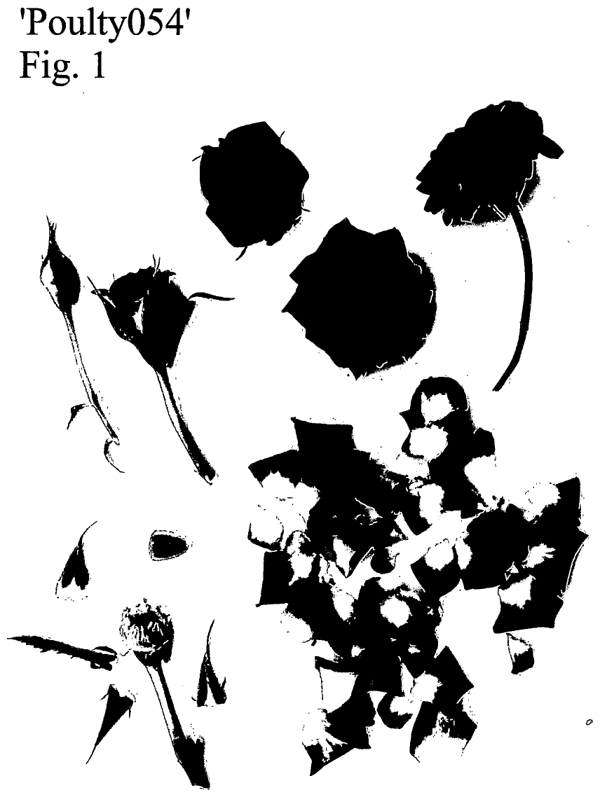

Specifically illustrated in FIG. 1 of the drawings are open flowers viewed from above and the side, flower petals and sepals detached revealing reproductive flower parts, a flower bud, and a partially opened flower.

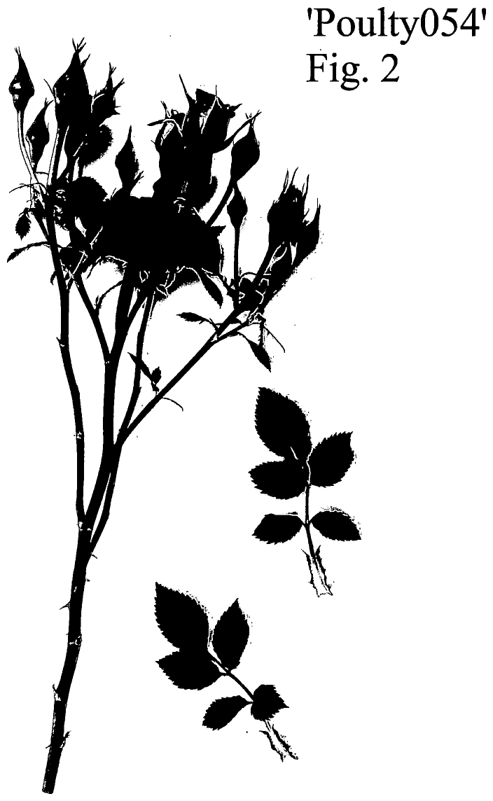

Specifically illustrated in FIG. 2 of the drawings is a cluster of flowers on a branch demonstrating the abundance of flowers on the plant, and mature leaves. Plants shown are 3 months of age.

### DETAILED DESCRIPTION OF THE VARIETY

The following is a description of ‘Poulty054’, as observed in its growth in an glasshouse nursery in Odense Denmark. Observed plants are 3 months of age, and were grown on their own roots in 17 cm pots. Color references are made using The Royal Horticultural Society (London, England) Colour Chart, 2001, except where common terms of color are used.

For a comparison, several physical characteristics of the rose variety ‘Poulpar108’, U.S. Plant Pat. No. 31,071, are compared to the claimed plant. The upper surface of outer petals of the claimed plant are Orange-Red Group N30B with an enlarged basal zone area of Yellow Group 9B. The upper surface of outer petals of ‘Poulpar108’ are Red 41B with a relatively small basal zone area of Yellow Group 10A. The comparison variety has greater expression of anthocyanin in the pedicel and sepals than the claimed plant.
Flower and flower bud:
   Blooming habit.—Continuous.
   Flower bud.—Size: Upon opening, about 20 mm in length from base of receptacle to end of bud. Bud diameter is 10 mm. Bud form: Ovoid. Flower and Flower Bud Bud color: As sepals divide petals are Yellow-Orange Group 18C with intonations of Red Group 41C. Sepal inner surface: Color: Yellow-Green Group 146D. Surface: Lightly pubescent. Sepal outer surface: Color: Yellow-Green Group 144A. Texture: Smooth. Sepal Shape: Apex: Cirrhose. Base: Flat at union with receptacle. Sepal Margin: Margins have very light foliaceous appendages on three of the five sepals. Sepal size: About 30 mm long, 9 mm wide. Receptacle: Texture: Smooth. Size: 10 mm in height, 9 mm wide. Color: Yellow-Green Group 144A. Shape: Funnel. Pedicel: Surface: Smooth. Length: About 20 to 30 mm. Diameter: 2 mm on average. Color: Yellow-Green Group 144B. Strength: Strong. Peduncle: Length: From 3 to 9 cm. Diameter: About 3.5 mm. Color: Yellow-Green Group 144A. Texture: Smooth.
   Flower bud development.—Flower buds are borne in clusters of about 15 flower buds per stem. Development resembles a cyme.
   Flower bloom.—Fragrance: Light. Duration: The blooms have a duration on the plant of approximately 21 days. Petals fall cleanly away from plant after flowers have fully matured. Size: Flower diameter is about 33 mm when open. Flower depth is14 mm. Flower shape: Open cup, double flower, with petals that curve out from the center. Shape of flower, side view: The upper portion is convex. The lower portion is deep concave.
   Petalage.—Under normal conditions, flowers have about 60 petals.
   General tonality of flower.—Open flowers are Red Group 52B and Red Group 40B.
   Petal color.—Upon opening, outer and inner petals. Upper surface: Basal zone and middle zone are Yellow Group 9B. Middle zone to margins are Orange-Red Group N30B. Lower surface: A large basal zone area of Yellow Group 12A. Marginal are Yellow-Orange Group 18B with shades of Orange-Red Group 33C. After opening, outer petals. Upper surface: A large basal zone area of Yellow Group 12B. Margins are Red Group 52B. Lower surface: Middle and outer zones are Yellow-Orange Group 19B with shades of Red Group 39B. Yellow Group 10A at the basal zone. After opening, inner petals: Upper surface: At basal zone Yellow Group 12B. Marginal zone is Orange-Red Group N32A. Lower surface: Yellow Group 12B at base. Margins Yellow-Orange Group 19C with shades of Red Group 39C.
   Petals.—Petal Reflex: Strong bilateral. Margin: Entire and uniform. No undulations of margin. Shape: Broad and elliptic. Apex shape: Rounded. Base shape: Acute. Size: About 16 mm (l)×18 mm (w). Texture: Smooth. Thickness: Average.
   Petaloids.—Size: 9 mm (l) by 5 mm (w). Quantity: 5 average. Shape: Elliptical with an acute base and rounded apices. Color: The upper surface is Orange-Red Group 32A with a basal zone petaloid spot of Yellow Group 12B. The lower surface is is Yellow-Orange Group 19C with shaded intonations of Red Group 39C. The undersurface has a basal petaloid spot of Yellow Group 12B.
   Reproductive flower parts.—Pollen: None observed. Anthers: Size: 2 mm in length. Color: Yellow Group 11C. Quantity: 30 on average. Filaments: Color: Yellow Group 9A. Length: 5 mm. Pistils: Length: 4 mm. Quantity: 20 on average. Stigmas: Color: Orange-White Group 159Styles: Color: Red Group 42A. Location of stigmas: Inferior in location relative to the length of the filaments and the height of the anthers. Hips: None Observed.
Plant:
   Plant Growth.—Upright. Plants are 30 cm in height, and 28 cm wide.
   Stems.—Color of juvenile growth: Yellow-Green Group 144A. Color of mature growth: Yellow-Green Group 144A. Length: Canes are about 13 cm from the base of the plant to the flowering portion. Diameter: About 5 mm. Internodes: On mature canes about 23 mm between nodes. Surface Texture: Young wood: Smooth. Older wood: Smooth.
   Long prickles.—Incidence: About 5 prickles per 10 cm of stem. Size: Average length of prickles on mature stems is 5 mm. Shape: Upper portion is linear. Lower portion is concave. Color: Juvenile prickles: Yellow-Green Group 144C. Mature prickles: Yellow-Green Group 144C.
   Plant foliage.—Compound leaf: 84 mm (l)×58 (w). Quantity: 3 to 4 leaves per 10 cm of stem on average. Leaf bearing angle to the stem: 45 degrees. Color of juvenile foliage: Upper side: Yellow-Green Group 144A. Lower side: Yellow-Green Group 144B. Color of mature foliage: Upper side: Yellow-Green Group 146A. Lower side: Yellow-Green Group 146B.
   Plant leaves and leaflets.—Stipules: Size: About 16 mm long, 3 mm wide. Quantity: 2 per compound leaf. Shape: Linear, slightly broad based with outward extending apices. Margins: Feather like, serrated. Color: Yellow-Green Group 144A. Petiole: Length: 20 mm average. Diameter: 2 mm. Upper surface color: Yellow-Green Group 144A. Lower surface color: Yellow-Green Group 144A. Rachis: Length: About 30 mm. Upper surface color: Yellow-Green Group 144A. Lower surface color: Yellow-Green Group 144A. Leaflet: Quantity: Normally 5 leaflets. Margins: Double serrate. Size: Terminal leaflets are about 36 mm long, 20 mm wide. Shape: Generally elliptical. Base: Rounded. Apex: Acute. Texture: Smooth. Thickness: Average. Arrangement: Odd pinnate. Venation: Reticulate. Glossiness: Not glossy. Disease resistance: Above average resistance to powdery mildew Sphaerotheca pannosa var. rosae, downy mildew Peronospora sparsa, rust Phragmidium spp., black spot Diplocarpon rosae, and Botrytis cinerea under normal growing conditions.
   Cold hardiness.—The variety is tolerant to USDA Cold Hardiness Zone 6.
   Heat tolerance.—The variety has been found to be suitable for climate conditions found in the American Horticulture Society heat zone 7.

I claim:

1. A new and distinct variety of rose plant named ‘Poulty054’ substantially as described and illustrated herein.
